(19) United States
(12) Patent Application Publication
(10) Pub. No.: US 2025/0130217 A1
(43) Pub. Date: Apr. 24, 2025

(54) Automatic Dissolution Testing System

(71) Applicant: Logan Instruments Corporation, Somerset, NJ (US)

(72) Inventors: Luke Lee, Belle Mead, NJ (US); Seth Michael Endy, Whitehouse Station, NJ (US); Manuel Villar, Fords, NJ (US)

(73) Assignee: Logan Instruments Corporation, Somerset, NJ (US)

(21) Appl. No.: 18/490,902

(22) Filed: Oct. 20, 2023

(51) Int. Cl. G01N 33/15; G01N 13/00; G01N 35/00; G01N 35/10

(52) U.S. Cl. CPC G01N 33/15; G01N 13/00; G01N 35/10; G01N 2013/006; G01N 2035/00346

(57) ABSTRACT

Dissolution testing system which uses a dissolution vessel to conduct a test of a tablet includes a frame on which the dissolution vessel is supported, a sampling probe movable into a position at least partly in an interior of the vessel, a temperature sensor on the sampling probe for sensing temperature of the dissolution media, a camera on the sampling probe for imaging dissolution of the tablet, and heating elements outside of the vessel interior for heating the vessel wall. A cover of the vessel has a media delivery conduit and a washing conduit passing therethrough. The vessel is in a housing that rotates between a testing position in which the vessel opening is oriented upward, and at least one additional position in which the vessel opening is oriented downward to allow for fluid flow out of the vessel interior, and drying and washing of the vessel interior.

### FIELD OF THE INVENTION

The present invention relates to an automatic or automated dissolution test system for testing dissolution of tablets and other pharmaceutical products.

The present invention also relates to various components of or used in an automatic dissolution test system for testing dissolution of tablets and other pharmaceutical products, such as a sampling probe, dissolution vessel, vessel washing device, and filter tip replacement device.

### BACKGROUND OF THE INVENTION

The tablet dissolution test is an FDA requirement and specificity by USP. It is an issue that such tests are very tedious. A typical dissolution tests start with media preparation in which test media is prepared to have a certain pH, and temperature-controlled and degassed under 6 PPM. It is necessary to precisely transfer the test media into dissolution vessels. The media temperature must be maintained at about 37 degrees Celsius during the tests to allow the tablets to dissolve at the normal human body temperature, and the dissolution samples must be withdrawn constantly as per the test protocols. After the test, all the tested media must be collected, and all the dissolution vessels must be washed for the next test. All such tests are in the pharmaceutical Quality Control (QC) departments 24/7 and depend on human operations, which creates many errors and is very time-consuming.

For example, laboratory personnel often complain about numerous difficulties with manually operated dissolution systems, from media preparation to dumping the tested media and washing the vessels, and also with respect to the transfer and analysis of the samples.

In the prior art, U.S. Pat. No. 3,802,272 (Bischoff et al.) describes an early apparatus for determining the dissolution rate of solid materials in liquids, and more particularly, automatic apparatus for determining dissolution rates on different solid materials. The apparatus includes dissolution test chambers connected via associated valves and tubing to an analyzer, means for operating the valves and tubing to supply a liquid sample from each chamber in a sequence to the analyzer and return the sample to its chamber, and means for back flushing the valves and tubing to minimize cross-interference between samples. The apparatus can also include additional groups of test chambers for automatically testing dissolution rates with a plurality of sets of sample material, and valve and control means for operatively coupling successive sets of chambers to the analyzer.

U.S. Pat. No. 6,336,739 (Lee) describes an air bath dissolution tester having a heating element within the USP specification dissolution test stirring shaft, easily attachable to a basket-type (USP) method I, or paddle (USP method II). This allows direct heat transfer to the test solution in the vessel, reducing heat up time. Temperature in the vessel is controlled by a controller responsive to test vessel temperature sensor. Temperature data is obtained during the test. The test vessel is located in a warm air chamber to prevent heat loss. The air chamber and test vessel are transparent, allowing the operator to determine the progress of the test. No water bath is required. The heated shaft promotes test solution degassing by direct heating and high-speed stirring during heat up, thus eliminating a separate step.

Other prior art in this field includes U.S. Pat. Nos. 3,109,913, 4,754,657, 4,856,909, 4,879,917, 4,924,716, 4,964,310, 5,011,662, 5,236,263, 5,412,979, 5,589,649, 5,639,974, 5,796,016, 5,816,701, 6,149,295, 6,303,909, 6,727,480, 7,543,354, 7,658,198, 7,914,741, 8,158,059, 8,511,148, 8,518,327, 8,903,673, 10,164,716, 10,486,119, 10,486,123, 10,576,435, and 11,197,939, and U.S. Pat. Appl. Publ. Nos. 20090207691 and 20100127980. All of the prior art mentioned herein is incorporated by reference herein to the extent the disclosure of any of these patents is needed to support the enablement of the present invention as disclosed herein.

### OBJECTS AND SUMMARY OF THE INVENTION

It is an object of at least one embodiment of the present invention to provide a new and improved dissolution testing system which includes one or more innovative components that simplify, automate, and/or expedite processes performed during the dissolution test.

In order to achieve this object, and possibly others, a dissolution testing system in accordance with the invention includes a frame on which a dissolution vessel is supported and which has an interior into which dissolution media and a pharmaceutical compound such as a tablet are placed to conduct a dissolution test of the pharmaceutical compound, and a sampling probe movable into a position at least partly in an interior of the vessel. There is a temperature sensor in connection with the sampling probe for sensing temperature of the dissolution media in the interior of the vessel when positioned below an upper surface of the dissolution media in the interior of the vessel, and a camera in connection with the sampling probe for imaging dissolution of the pharmaceutical compound during the dissolution test.

Heating elements are provided outside of the interior of the vessel for heating a wall of the dissolution vessel to thereby cause the dissolution media in the interior of the dissolution vessel to be heated. A media delivery conduit is on the frame for delivering dissolution media to the interior of the vessel, along with a washing conduit for delivering washing media to the interior of the vessel to wash the interior of the vessel. The vessel is mounted in a rotatable vessel housing that operatively rotates between a first, testing position in which an opening of the vessel is oriented upward, and at least one additional position in which the opening is oriented downward to allow for fluid flow out of the interior of the vessel.

As to the sampling probe, it includes a hollow shaft, and the temperature sensor is in a first end of the shaft. The camera is also at the first end. The sampling probe also includes an electrical slip ring at a second end of the shaft opposite to the first end and configured to avoid tangles of at least one wire leading to the temperature sensor and the camera at a top of the shaft.

The heating elements may include a film heater below the dissolution vessel, a ceramic heater around a lower portion of the dissolution vessel, and another ceramic heating around an intermediate portion of the dissolution vessel above the other ceramic heater. An overheat or temperature control sensor senses temperature of the wall of the dissolution vessel and controls heating provided by the heating element to prevent overheating.

The system may include a self-adjusting vessel cover configured to freefall onto the dissolution vessel. The media delivery conduit and washing conduit extend through the cover and terminate in a nozzle angled toward a side of the vessel. There may be a tablet dropping tray from which tablets are dropped into the interior of the vessel above the cover.

The system also preferably includes a vessel housing rotation mechanism coupled to the vessel housing and that rotates the vessel housing between the different positions. The rotation mechanism includes a motor having an output shaft, a gear mounted to the vessel housing, and a transmission belt that transfers rotational motive force from the output shaft through the transmission belt to the gear. The other positions include an emptying position in which the opening of the vessel is to one side, a washing position in which the vessel is upside down and the opening of the vessel is downward, and a drying position in which the opening of the vessel is angled downward. There may be a washing device for cleaning the interior of the vessel when in the washing position using an extendable rotation nozzle arranged below the vessel. The washing device also includes a telescoping extension mechanism coupled to the nozzle to extend upward upon flow of water through the rotation nozzle, and a screen tray around the nozzle. There may also be a drying device that dries the interior of the vessel when the vessel is in the drying position.

To improve basket usage, there may be a rotation carousel on the frame that has basket compartments each having an upper part for storing a respective, clean basket, a lower part configured to retrieve a respective, used basket and an intermediate part including at least one gasket. To provide a new basket on the shaft, the shaft is movable into engagement with one of the baskets when in the upper part of a respective compartment until such movement is resisted by the at least one gasket. To remove a used basket from the shaft, the shaft is movable into the lower part of a respective compartment until such movement is resisted by the at least one gasket and then moved out of the lower part of the respective compartment. The used basket remains in the lower part of the respective compartment, and after which the carousel is rotated to enable the shaft to engage with a new basket in another one of the compartments.

The system may also include a rotatable tablet loading device arranged above the vessel and including a rotation carousel configured to contain from six to twelve tablet compartments arranged around a periphery of the carousel. A gate is below the carousel and through which a tablet falls. The gate is controlled relative to rotation of the carousel to control dropping of tablets into the vessel.

An automatic filter tip replacement device in the system includes a movable filter track that receives a row of filter tips and is situated above the vessel, and a gate below the filter track. The gate has a closed position in which a sampling probe is moved into engagement with one of the filter tips on the filter track and an open position in which the sampling probe passes through the gate with an engaged filter tip into the interior of the vessel. The filter track is moved to alternatingly bring each of the filter tips into a position to be engaged by the sampling probe.

A sample tray transfer station in the system includes a movable robot arm, a sample tray storage rack on one side of the robot arm in which a plurality of storage racks are stored, and a sample collector on another side of the robot arm in which samples are collected. The robot arm is controlled to individually place one of the storage racks from the sample try storage rack onto the sample collector to collect samples, and remove each of the storage racks from the sample collector after samples are collected and move it to the sample tray storage rack.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1 is a perspective view of a dissolution testing system in accordance with the invention;

FIG. 2 is a perspective view of a shaft used in the dissolution testing system shown in FIG. 1;

FIG. 3 is a side view of a dissolution vessel used in the dissolution testing system shown in FIG. 1;

FIG. 4 is a perspective view of the bottom of the vessel shown in FIG. 3;

FIG. 5 is front view of dissolution vessels and a tablet dropping tray of the dissolution testing system shown in FIG. 1;

FIG. 6 is side view of one of the dissolution vessels;

FIG. 7 is a perspective view of the vessel housing of the dissolution testing system shown in FIG. 1;

FIG. 8 is a side view of the vessel housing in an operating position;

FIG. 9 is a side view of the vessel housing in a pouring position;

FIG. 10 is a side view of the vessel housing in a drying position;

FIG. 11 is a side view of the vessel housing in a washing and cleaning position;

FIG. 12 is a perspective view of a washing device of the dissolution testing system shown in FIG. 1;

FIG. 13 is a side view of the washing device in an extended position in which it is able to clean the interior of a dissolution vessel of the dissolution testing system shown in FIG. 1;

FIG. 14 is a side view of the washing device in a retracted, rest position;

FIG. 15 is a view of the housing of multiple dissolution vessels when in the washing and cleaning position;

FIG. 16 is a view showing the dissolution vessel in a drying position;

FIG. 17 is a perspective view of the rotation carousel for baskets of the dissolution testing system shown in FIG. 1;

FIG. 18 is a top view of a tablet loading device of the dissolution testing system shown in FIG. 1;

FIG. 19 is a top of a filter tip replacement device of the dissolution testing system shown in FIG. 1;

FIG. 20 is a perspective view showing the filter track of the dissolution testing system shown in FIG. 1;

FIG. 21 is an enlarged view of part of the filter tip replacement device of the dissolution testing system shown in FIG. 1; and

FIG. 22 is a perspective view of a sample tray transfer station of the dissolution testing system shown in FIG. 1.

### DETAILED DESCRIPTION OF THE INVENTION

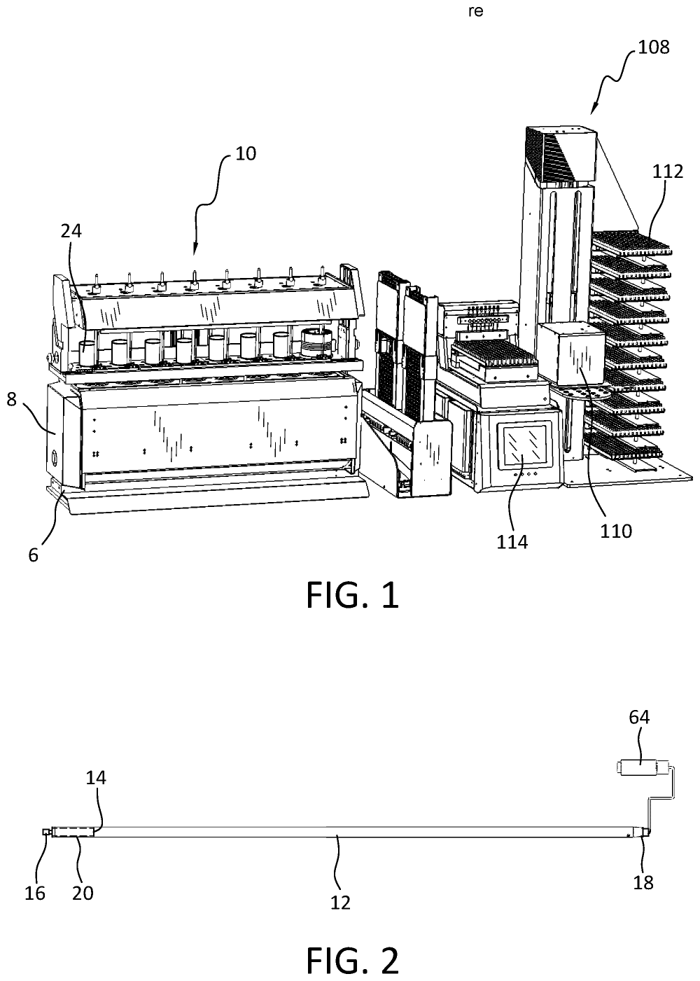

Referring to the accompanying drawings wherein the same reference numbers refer to the same or similar elements, FIG. 1 shows a fully automated dissolution testing system 10 in accordance with the invention, and is intended to be marketed commercially by the applicant and assignee as the Logan System 2400. Dissolution testing system 10 has several stand-alone components that are operatively arranged side-by-side on a horizontal support surface and provides significantly improved dissolution testing in comparison to prior art systems.

Automated dissolution testing system 10 is configured to be able to conduct ten batches in each testing procedure in accordance with USP 1 or 2 from media delivery to analysis unattended, i.e., without manual involvement. It is thus very advantageous that all stages of the dissolution process using the dissolution testing system 10 are computer-coordinated and carried out entirely without user intervention. Dissolution testing system 10 can also be constructed to conduct a different number of batches.

Dissolution testing system 10 precisely delivers, preheats, and degasses media into eight dry-heat test vessels 24 arranged in a main component to the far left in FIG. 1 which has a frame 6 and a housing 8 thereon, removing the added complication of a water bath for heating the vessels. Each vessel 24 preferably has one or more associated cameras to record the dissolution for subsequent viewing and result verification. After the test is over, the vessels 24 are controlled to automatically empty the media, they are then sprayed, washed, and blow-dried, ready for the next batch(es) of test samples to provide the total of ten batches. The number of dissolution vessels 24 may be different than eight as shown in the illustrated embodiment.

Dissolution testing system 10 is preferably equipped with two types of filter changers. One version includes an automated filter tip changer for online ultraviolet (UV) analysis. An additional membrane filter changer is available for sample collection for offline HPLC analysis.

Dissolution testing system 10 includes several innovations described below. These innovations are ideally provided in combination with one another to provide an optimal dissolution testing system. Nevertheless, a dissolution testing system in accordance with the invention is not required to include all the innovations disclosed below, and moreover, each of the innovations can be considered an independent invention and can be used in different dissolution testing systems or other systems wherein the innovation is found to be useful. The disclosure herein therefore does not limit in any manner the intended uses of the devices disclosed herein, nor possible combinations of such devices.

Dissolution testing system 10 includes a sampling probe 12 including an elongate shaft 22, a temperature sensor 14 and a camera 16 (that can be used together or used separately) with a slip ring 18 for wire connection (FIG. 2). Sampling probe 12 is intended to address a problem in existing dissolution testing systems, namely that the user must manually insert the sampling probe into the media (in the vessel) to measure the temperature. Not only does the user need to measure six to eight vessels individually before the test starts, but the user must also constantly measure the temperature during the test to make sure that the temperature stays at about 37 degrees Celsius. Also, the user measures the temperature when collecting the sample. This process requires numerous manual temperature measurements which is time-consuming and labor consuming.

In some cases, the temperature sensor in existing dissolution testing systems is mounted in the sampling manifold, which only allows the temperature measurement of the media at certain time intervals. Further, in terms of USP 1 (basket method), there is no way to observe the dissolution status through the basket. This problem creates the uncertainty of the test.

Dissolution testing system 10 addresses these issues by providing for each dissolution vessel 24, the sampling probe 12 as shown in FIG. 2 that includes three main elements: the temperature sensor 14 on the side of a cylinder-shaped fixture 20, the camera 16 at the bottom of the shaft 22 for tablet dissolution monitoring, and the electrical wire or slip ring 18 to avoid wire tangles at a top of the shaft 22. The shaft 22 is hollow between closed end regions.

Temperature sensor 14 is fixed on the side of the stainless steel fixture 20 which tightly fits in the shaft 22 so that the temperature sensor 14 is firmly connected with the inner side of the shaft 22. Since the sampling probe 12 is in the dissolution media in the vessel 24 during the test, i.e., below the upper surface of the dissolution media in the vessel 24, this design can measure the temperature of the dissolution media accurately and constantly. Alternatively, the temperature sensor 14 may be part of a media temperature sensor ring pressed in from the lower part of the shaft 22. The temperature of the fluid in the vessel can be read constantly by temperature sensor 14, or at whatever intervals are desired or required.

Camera 16 is inserted into the same fixture 20 as the temperature sensor 14 to image and enable recordation of the dissolution status of the tablet in the dissolution medial in the vessel in real time during the test. Camera 16 may be considered a miniature camera as this term is used and/or known by those skilled in the art to which this invention pertains. Use of the camera 16 is optional. This camera 16 is especially suitable for the basket method, which enables the observation of dissolution of the tablet in the dissolution vessel from the inside of the basket. This design solves the problem of difficult observation of the dissolution process.

Temperature sensor 14 on the fixture 20 in the shaft 22 is a preferred manner to sense temperature of the dissolution media but the invention is not limited to the use of such a temperature sensor 14. Rather, at a minimum, the dissolution testing system 10 includes temperature sensing means in connection with the sampling probe 12, but not necessarily on the fixture 20 inside the shaft 22 of the sampling probe 12, for sensing temperature of the dissolution media in the interior of the vessel 24 when positioned below an upper surface of the dissolution media in the interior of the vessel 24. These temperature sensing means may be other structure that achieves the same temperature sensing result as temperature sensor 14.

Similarly, camera 16 on the fixture 20 in the shaft 22 is a preferred manner to image the dissolution occurring in the vessel 24, but the invention is not limited to the use of such a camera 16. Rather, at a minimum, the dissolution testing system 10 includes imaging means in connection with the sampling probe 12, but not necessarily in the shaft 22 of the sampling probe 12, for imaging dissolution of the pharmaceutical compound during the dissolution test occurring in the interior of the vessel 24. These imaging sensing means may be other structure that achieves the same imaging result as camera 16.

The slip ring 18 is installed on the side of the shaft 22. The slip ring 18 and an aviation plug 64 are held in place by springs between the aviation plug 64 and the slip ring 18 to prevent the wires from twisting or breaking while rotating. Slip ring 18 solves the problem of the wire following the rotation of the shaft 22. The top of the shaft 22 is thus able to rotate using the slip ring 18, which allows the sensor wires (that connected to the temperature sensor 14 and camera 16 at the bottom region of the shaft 22) to rotate with the shaft 22.

The bottom of the shaft 22 has an internal thread so that the external thread of an insert, e.g., a paddle or basket head, can be connected threadingly to the shaft 22 to operatively cause stirring of the dissolution media in the vessel.

In a preferred embodiment, dissolution testing system 10 is a dry heat dissolution tester which avoids problems with prior art dissolution testing systems. In some conventional dissolution testing, the dissolution vessels are heated with the water bath, which requires manual filling and changing of the water in each bath, and increases the complexity of the operation. Other disadvantages are that it is also possible that the water heat circulator can overheat when the water level is low, the water can leak from the water tank or piping, and the water can be contaminated in a few days.

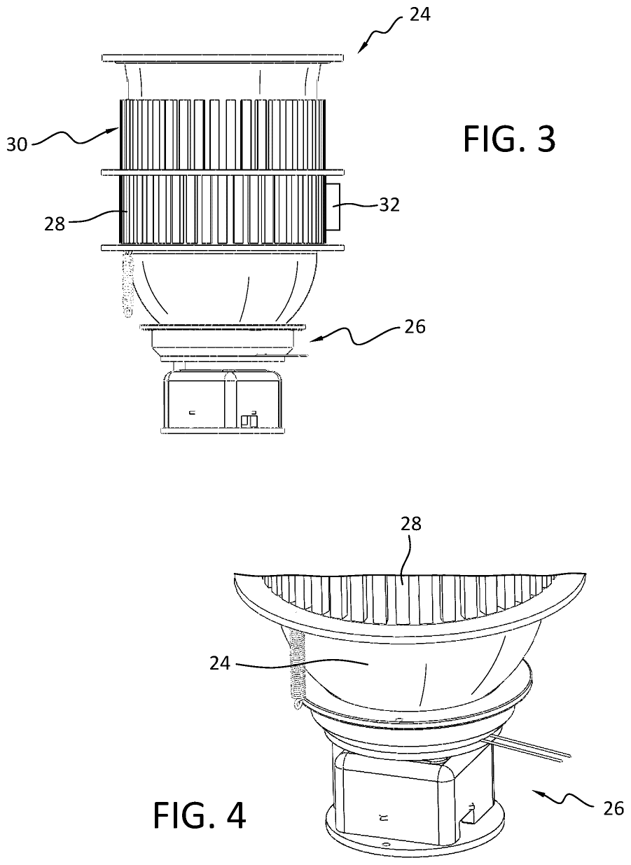

To address and avoid these issues, the dissolution vessels 24 operate based on a dry heat principle in that each includes three or more heating elements or heaters and temperature control sensors (FIG. 3). The heating elements or heaters will generally be referred to as heating means. A preferred, but not limiting, embodiment of the heating means is shown and include a first, lower heating element 26 located on or below a bottom of the vessel 24, a second, intermediate heating element 28 around the vessel 24 and parallel to the area inside the vessel 24 which about 500 ml of solution will be situated in the vessel 24, and a third, upper heating element 30 around the vessel 24 and parallel to the area in which about 500 ml of solution will be situated in the vessel 24 (above the 500 ml of solution to be heated by heating element 28). A safety or temperature control sensor 32 is provided to prevent overheating (FIG. 3). The heating elements may be tightly wrapped around the vessel 24. This unique design of heating elements makes the camera recording available. Sensor 32 may be mounted between the lower heating element 28 and the bottom of the vessel 24.

Heating element 26 is preferably ring-shaped (FIG. 4) The intermediate and upper heating elements 28, 30 are tubular and each surrounds a discrete region of the vessel 24 in which about 500 ml of dissolution media is situated when present in the interior of the vessel 24. This estimated heating volume may vary depending on the vessel size and volume. The objective of the multiple heating elements is that each preferably heats a different area of the vessel 24, and focus on heating only the dissolution media in the interior of the vessel alongside the respective heating element.

There are thus multiple heating sources of the dissolution media in the vessel 24 during use that can be controlled by a controller or processor in the housing 8 on the frame 6 of the dissolution testing system 10 to provide a desired heating effect. This same controller or processor, or more generally control unit, includes electronic components typical of dissolution testing systems and is coupled to the components it controls, from which it is provided data, from which it receives manually-entered commands, etc., by electrical or wireless connections. The processor is not shown as it is situated in the housing 8, but may be represented by a block connected by lines (indicating wires or wireless connections) to the other components of the dissolution testing system 10 disclosed herein. One skilled in the art to which this invention pertains would be able to provide such a processor and connect it to the other components of the dissolution testing system 10, and provide for control features to enable operation of the dissolution testing system 10 in the desired manner using the innovative components disclosed herein in view of disclosure herein.

Heating elements 28, 30 may be ceramic heaters around the respective cylindrical portion of the vessel 24 and heating element 26 may be a film heater on the bottom of the vessel 24, i.e., on or below the hemispherical portion of the vessel 24. Optionally, there is a camera at the bottom of the vessel 24.

Another notable feature of the dissolution testing system 10 is that it includes a self-adjusting vessel cover with the media delivery nozzle and the washing nozzle. Before the dissolution test conducted using existing dissolution testing systems, the user must preheat, degas, and precisely transfer the dissolution media into the dissolution vessel. This process needs to be repeated for up to twelve vessels. After the dissolution test is over, the user must clean each sampling probe in preparation for the next test. During the dissolution tests, the media evaporation is a big problem also.

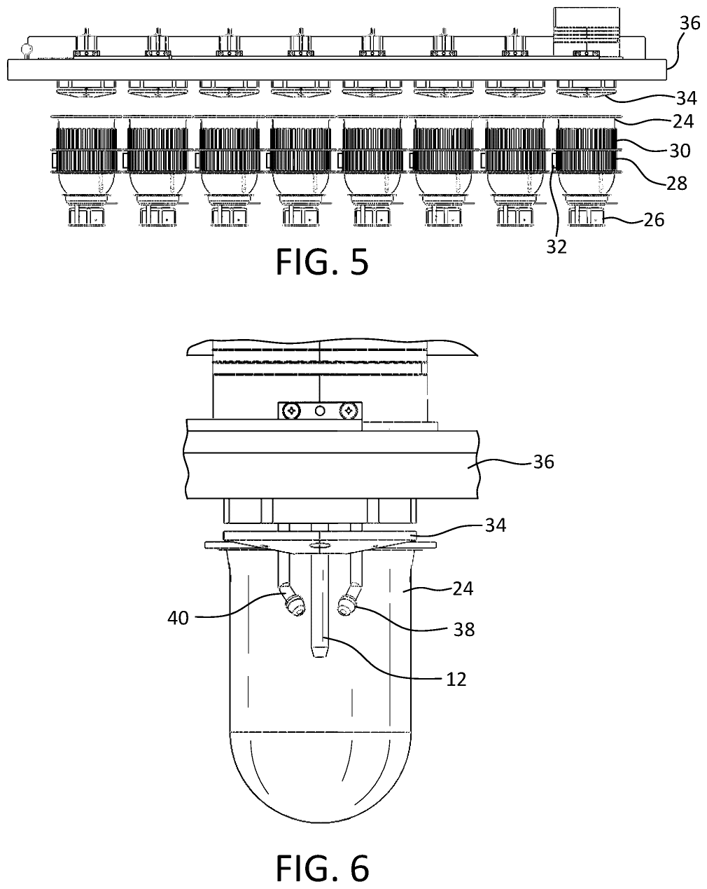

Dissolution testing system 10 provides a solution to these issues by providing a hanging vessel cover 34 that is mounted under a tablet dropping tray 36 extending over all of the vessels 24 from which tablets are dropped into the vessels 24 during a test (FIGS. 5 and 6). During the test, each vessel cover 34 freefalls onto the respective vessel 24 by the effect of gravity. Each vessel cover 34 has a curved lower surface and is configured to seal against the entire peripheral surface of an upper region of the respective vessel 24. This allows the vessel covers 34 to firmly cover the dissolution vessels 24.

A media delivery nozzle 38 and washing nozzle 40 are mounted to each vessel cover 34 to extend below the lower surface thereof (FIG. 6). Each media delivery nozzle 38 is preferably aimed at the respective vessel wall to avoid bubbles during media filling. Each washing nozzle 40 is preferably aimed at the respective sampling probe 12. The water is sprayed from the washing nozzles 40 to clean the sampling probe 12. Conduits and associated structure to provide media to the media delivery nozzles 38 and washing fluid to the washing nozzles 40 are known to those skilled in the art to which this invention pertains.

Media delivery nozzles 38 extending through the covers 34 are a preferred manner to delivery dissolution media to the vessels 24 for testing purposes but the invention is not limited to the use of such media delivery nozzles 38. Rather, at a minimum, the dissolution testing system 10 includes media delivery means on the frame 6 for delivering dissolution media to the interior of the vessels 24, but not necessarily extending through the covers 34. These media delivery means may be other structure that achieves the same media delivery result as media delivery nozzles 38.

Similarly, washing nozzles 38 extending through the covers 34 are a preferred manner to deliver washing fluid to wash the inside surfaces of the vessels 24 between tests but the invention is not limited to the use of such washing nozzles 38. Rather, at a minimum, the dissolution testing system 10 includes washing means on the frame 6 for delivering washing fluid to the interior of the vessels 24, but not necessarily extending through the covers 34. These washing means may be other structure that achieves the same washing fluid delivery result as washing nozzles 38.

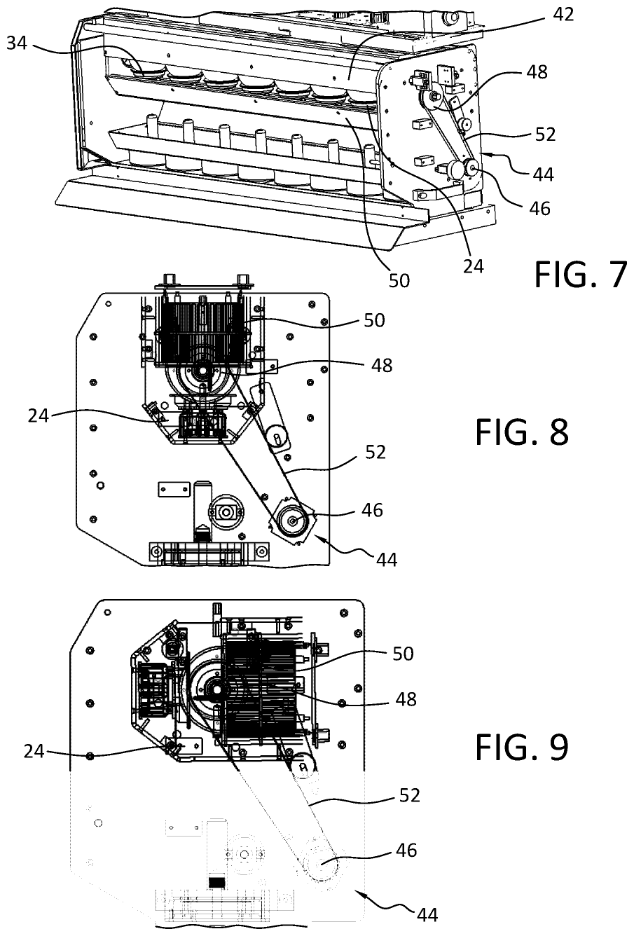

Another feature of dissolution testing system 10 is the presence of a rotation washing and cleaning vessel chamber 42 (FIG. 7). In conventional dissolution testing systems, to empty the dissolution vessels, the user must remove the vessel from the test position, empty the vessel, clean the vessel, and wait for it to dry. Only one vessel can be cleaned manually per person at a time, which is time-consuming and labor-intensive.

To expedite the dissolution vessel emptying, cleaning and drying process, in the dissolution testing system 10, the dissolution vessels 24 are housed in a light protection chamber 42 (FIG. 7). There could be anywhere from, for example, six to eight dissolution vessels 24 in the chamber 42. The dissolution testing system 10 is provided with a chamber rotation system that is configured to enable all of the vessels 24 in the chamber 42 to rotate up to 360 degrees at the same time, by rotating the housing 50 of the chamber 42 to which the vessels 24 are fixed, e.g., to a side wall thereof. The chamber rotation system 44 includes a motor having an output shaft 46, a gear or pulley 48 mounted to a housing 50 of the chamber 42 and a belt 52 that transfers the rotational motive force from the output shaft 46 through the belt 52 to the pulley 48 to cause rotation of the housing 50. The position of the gear 48 is selected to provide a suitable rotation axis for the housing 50 of the chamber 42 to achieve the various positions.

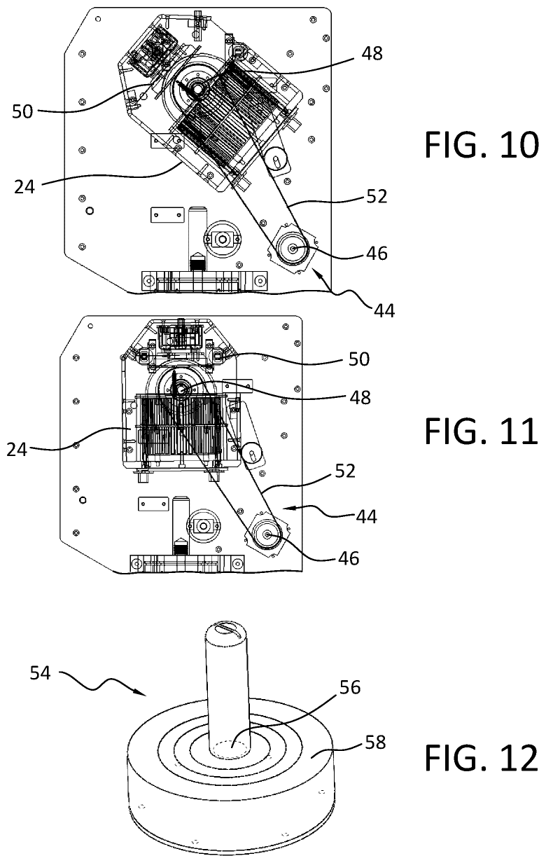

During this rotation, the chamber 42 carries the vessels 24 therein to and from several positions of use, namely, the filling and testing position in which the opening of each vessel 24 is upward and the test is performed (FIG. 8), the pouring or emptying position in which the opening of the vessels 24 is to one side and any contents in the vessels flow out of the vessels 24 (FIG. 9), the drying position in which the opening is angled downward and the interior of the vessels 24 is dried by a drying device (FIG. 10), and the washing and cleaning position in which the vessels 24 are upside down and the opening of the vessels 24 is downward so that any water entering the vessels 24 during the washing process freefalls downward out of the vessels 24 (FIG. 11).

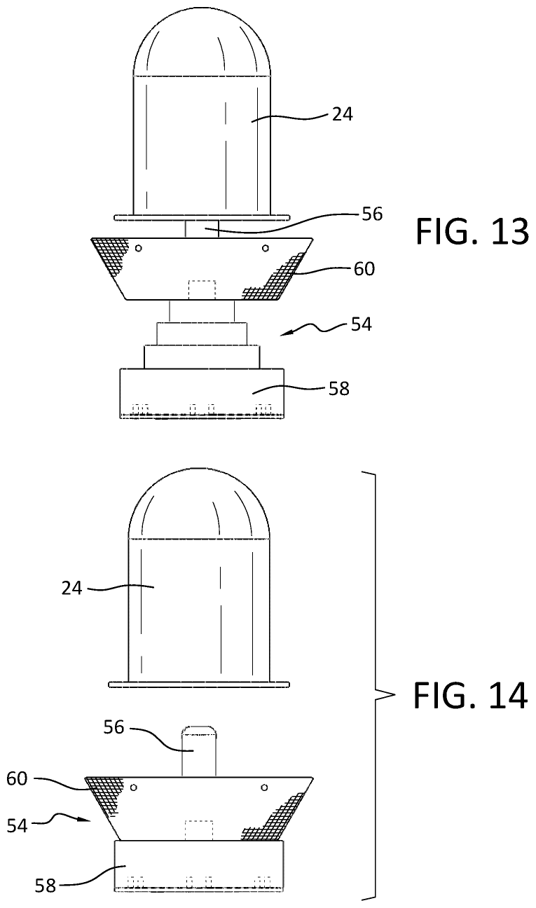

When the vessels 24 are rotated to the washing position, shown in FIG. 11, water is directed into the interior of each vessel 24 by a respective washing device 54 and cleans the vessel 24 (FIG. 12). When the test is over, the rotation vessel chamber 42 rotates 180 degrees from the test position (FIG. 8) to the pouring or emptying position (FIG. 9) to pour the media from the vessels 24. A water pump (not shown) pumps the water used to wash the vessels 24 into an extendable rotation nozzle 56 of each washing device 54 (FIGS. 12-15). The water pressure forces the extendable rotation nozzle 56 to pop up via a telescoping extension mechanism 58 into the interior of the vessels 24 to provide the position shown in FIG. 13, and the washing cycle starts.

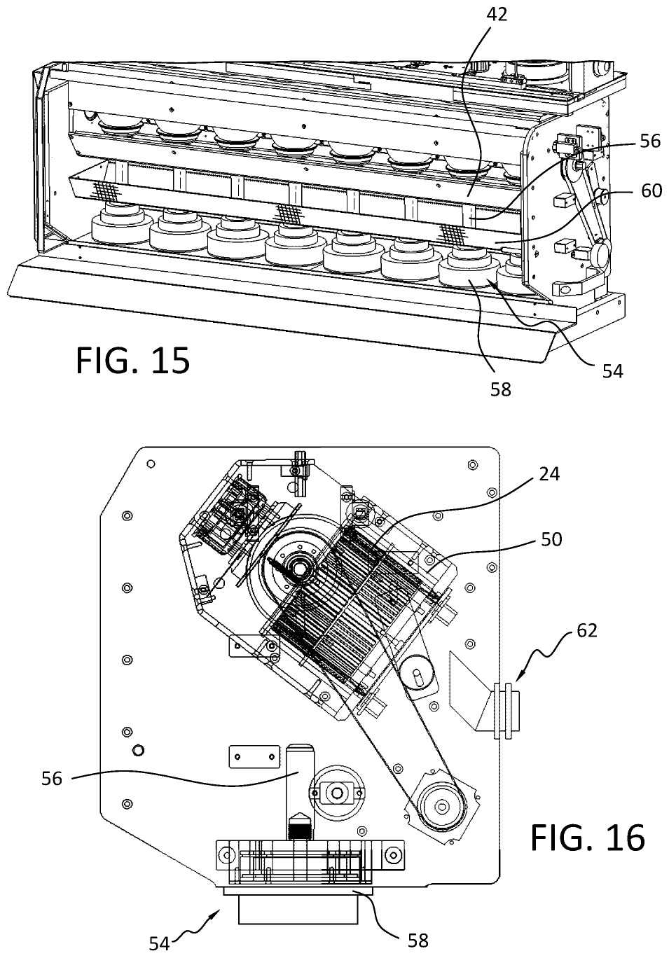

A computer or processor in the dissolution testing system 10 (not shown) controls the wash time to ensure the vessels 24 are cleaned. A single screen tray 60 is associated with all the nozzles 56 to catch disposable items and large particles to avoid clogging the drain of the dissolution testing system 10 (FIGS. 14 and 15). When the washing is completed, there is no more water pressure and the weight of the screen tray 60 returns all nozzles 54 to the home position (FIG. 14 wherein the telescoping extension mechanism 58 is in its rest state).

In conventional dissolution testing systems, after cleaning the vessels, the user must hang the vessels and wait for them to dry, which takes a long time. Also, dirt can get into the vessels when hanging, which negatively impacts the test and thus often requires re-washing and re-drying.

Dissolution testing system 10 overcomes these drawbacks by providing a blowing device 62 in the housing of the dissolution testing system that blows hot air into the vessels (FIG. 16). After the vessels 24 are washed, the vessel rotation chamber 42 is rotated to bring all the vessels 24 therein to the drying position (FIG. 10). A respective high-power blower used as the blowing devices 62 forces air into the vessels 24 to blow out all waterdrops. Meanwhile, the dry heat element heats the vessels 24 to evaporate any moisture that accumulated on the vessel wall.

Yet another aspect of the dissolution testing system 10 is that it allows for an automatic basket and shaft engagement device. In conventional dissolution testing systems, to do the basket method, the pharmaceutical tablet needs to be placed in the basket and manually set onto the shaft. At the end of the dissolution, the basket needs to be manually removed, cleaned, and replaced. Only one basket can be done at one time per person. Besides, the basket is fragile, so manual handling can damage the basket during the attaching and removing process.

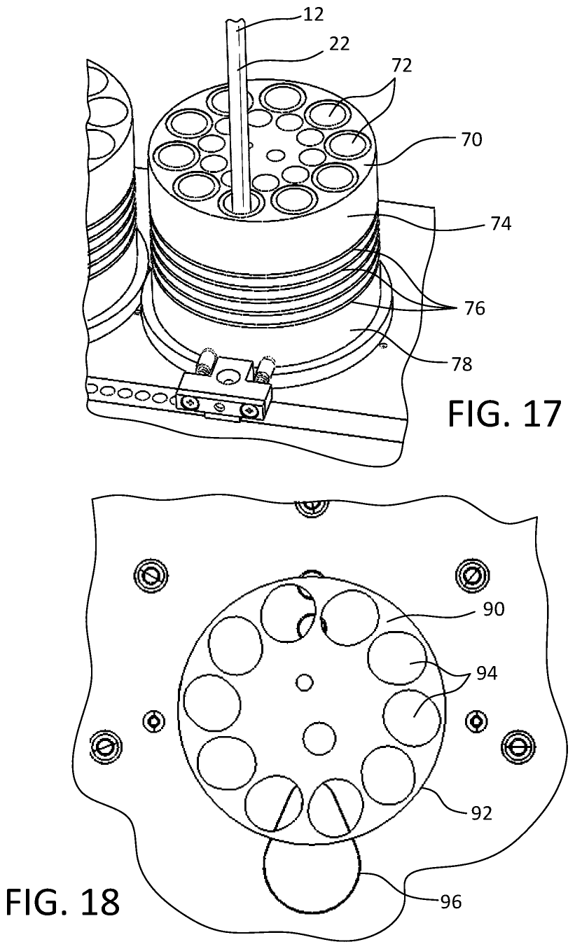

To address these issues, dissolution testing system 10 includes a rotation carousel 70 that contains six to twelve basket compartments 72 (FIG. 17). Carousel 70 is arranged on the frame 6 and may be in the housing 8. An upper part of the compartment 72 is for storing new, unused baskets 74. A middle part of each compartment 72 contains several layers of rubber gaskets 76, although one could also suffice. Gaskets 76 have holes below each stored basket 74 that are smaller than the basket 74 so that the basket 74 cannot fall through the gaskets 76. A lower part 78 of the compartment 72 is for basket retrieval.

In operation to position a basket on the shaft 22 of the sampling probe 12, a dissolution tester drive head (not shown) brings the shaft 22 down toward the carousel 70 into one of the compartments 72 that contains a clean basket 74. Once the head of the basket 74 is engaged on the shaft 22, the gaskets 76 create resistance. The resistance pushes the basket 74 onto the shaft 22. Each layer of the gaskets 76 creates resistance to make sure the basket 74 is firmly attached to the shaft 22. Then, the basket 74 and the shaft 22 are lifted and re-positioned and descend into the vessel 24 to start the dissolution test. The sampling probes 12 are manipulating to engage with the baskets 74, one for each vessel 24.

When the test is done, after the washing cycle, the drive head brings the basket 74 and the shaft 22 upwards. When drive head reaches the basket retrieval compartment 72, the gaskets 76 pull the basket 74 away from the shaft 22 and keep the basket 74 in the basket retrieval compartment 72. The carousel 70 advances one position for the next basket test. The used baskets are retrieved through a door at the front of the testing system 10, which is opened to access the used baskets, and also to place new baskets into the upper part of the compartments 72. The mechanical structure that connects to and manipulates the drive head may be as in any conventional system, with the movement of the drive head now being controlled to engage with the carousel 70. Thus, the carousel 70 could be installed in any conventional dissolution testing system and expedite the basket manipulating thereof.

Another innovation in dissolution testing system 10 is a rotatable tablet loading device 90 (FIG. 18). This innovation is designed to address the problem with existing dissolution tests performed using conventional dissolution testing systems in that they require manual dosing. Capsules or tablets must be manually placed into the sinker before dropping into the vessel. After the dissolution test, it is necessary to manually take out the sinker, and clean and replace it. Also, one operation can only drop one tablet, so this process needs to be repeated six to eight times before the start of the test in a short period of time.

Rotatable tablet loading device 90 is arranged on the frame 6 in the housing 8 and includes a rotation carousel 92 that contains from six to twelve tablet compartments 94 arranged around the periphery of the carousel 92, although the number is not essential in the invention. The compartments 94 can be equiangularly spaced around the periphery and the number of compartments 94 can depend on the diameter of the carousel 92. There is a gate 96 under the carousel 92 and the gate is configured to open when the carousel 92 is ready to drop the tablet. As such, six to eight tablets can be dropped into the vessel 24 at the same time. The gate 96 automatically closes when tablet dropping is finished to avoid moisture getting into the carousel 92. Each time a test is completed, the rotation carousel 92 automatically rotates, getting ready for the next test. The movement of the gate 96 may be controlled by control means such as processor or computer of the dissolution testing system 10.

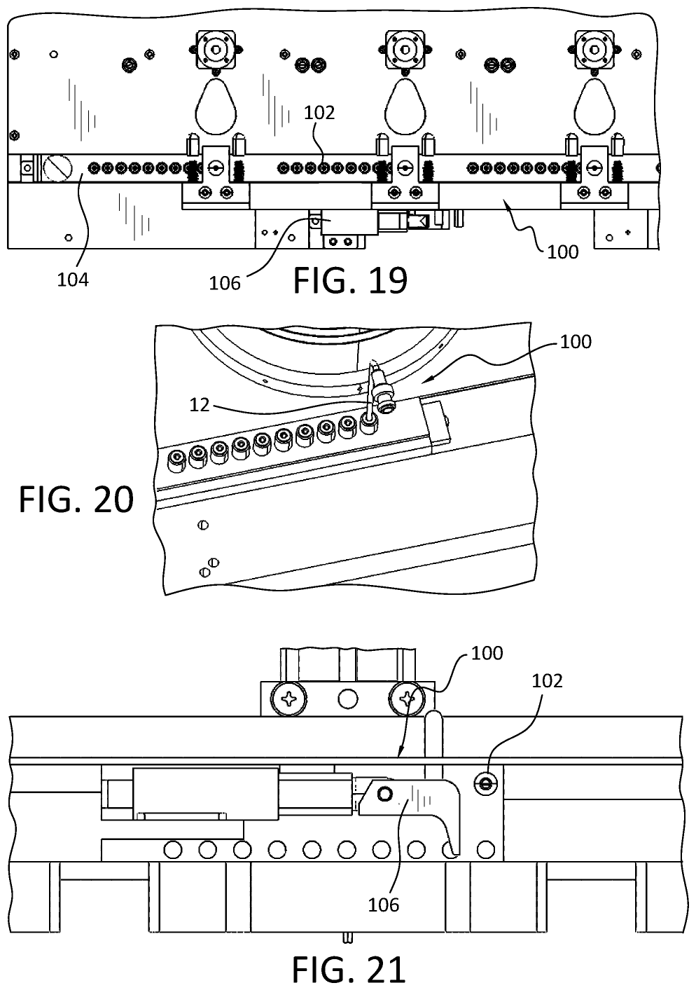

Dissolution testing system 10 also includes an automatic filter tip replacement device 100, shown in FIGS. 19-21, that is also arranged on the frame 6 in the housing 8. This device is designed to address a problem in conventional dissolution testing systems wherein during the dissolution test, particles are released from the tablets but then when collecting samples, particles might clog the sampling line. This can also create the sample analysis problem. To solve this problem using conventional dissolution testing systems, the user must manually install a filter on the tip of the sampling probe before taking the sample from the dissolution vessel. This process must be repeated six to twelve times for one dissolution running, which is time consuming. Also, the filter tip is easily broken by human handling.

For automatic filter tip replacement device 100, a row of 6-12 filter tips 102 is mounted on the filter track 104 that is under the sampling probe. There is a gate below the filter tip 102. Before the test, the motorized sampling manifold drop down all sampling probes. The sampling probe goes down and engages the filter tip 102 when the gate is closed. This process makes filter tip 102 firmly attached to the sampling probe.

After the engagement, the gate opens that allows the sampling probe with filter tip 102 into the dissolution media for sample filtration. When the dissolution test is over, the sampling manifold goes up, bringing the filter tip 102 through the filter discharge device, and makes the used filter tip drop into the vessel. After this, the filter tip 102 is caught by the screen tray when emptying the media (as described above). When the cycle is completed, the filter track push bar 106 advances one position to push all filter tips 102 forward to the filter engagement position, waiting for the next test. This process is repeated until all filter tips 102 are used. After all batches are completed, the user can reset the filter track 104 to the home position and reload filter tips 102 for the next dissolution batch.

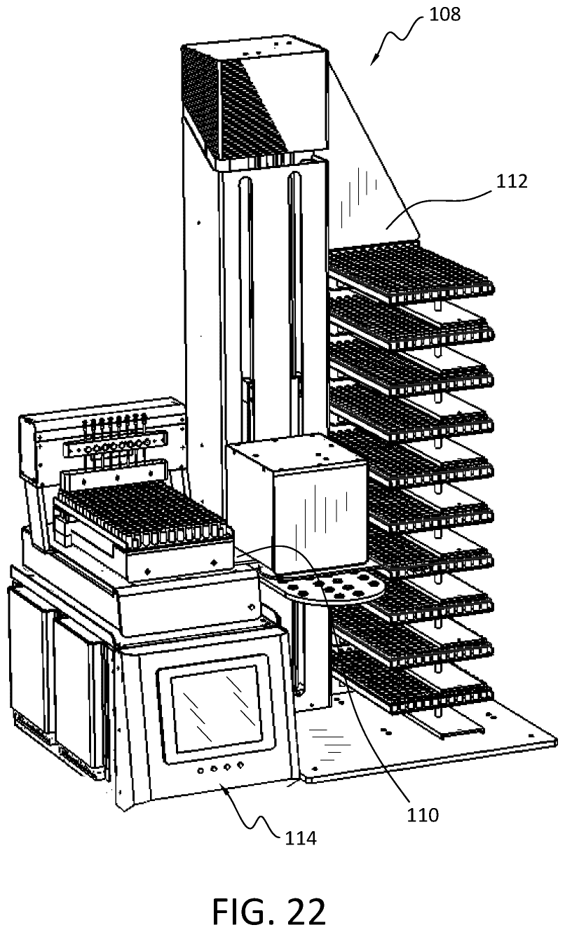

The final innovation in dissolution testing system 10 relates to a sample tray transfer station (FIGS. 1 and 22). For the dissolution test using conventional dissolution testing systems, the user must place vials into the sample tray and place the sample tray on the sample collector. After the dissolution test is over, the user must manually remove the sample tray from the sample collector and take sample vials for further analysis. The user repeats this process for each dissolution test run.

To avoid the inherent limitation on sample transfer in view of the need for manual action, dissolution testing system 10 includes a sample tray transfer station 108 having a computer-controlled robot arm 110 which is configured to move up and down, left and right up to 270 degrees. The structure to control and enable such movement may be any conventional structure. A sample tray storage rack 112 is placed on one side of the robot arm 110, and stores a set number of sample trays for use in the dissolution testing by system 10. A sample collector 114 of the dissolution testing system 10 is placed on another side of the robot arm 110. Sample tray storage rack 112 may be a conventional sample tray storage rack and is preferably one manufactured and sold by the applicant herein. Sample collector 114 may be a conventional sample collector and is also preferably one manufactured and sold by the applicant herein. As shown, it includes a control panel for controlling the sample collector 114.

When dissolution testing by dissolution testing system 10 starts, the robot arm 110 takes the first tray on top of the sample tray storage rack 112 and transfers the tray onto the sample collector 114 to collect samples during the test. When the dissolution run is over, the robot arm 110 transfers the tray back to the sample tray storage rack 112 and takes another or second sample tray from the sample tray storage rack 112. This process repeats from the top to bottom of the sample tray storage rack 112 until all sample trays present in the sample tray storage rack 112 are used.

The arrangement of the controllable robot arm 110 and sample tray storage rack 112 can be situated next to the sample collector of existing dissolution testing systems to automate the sample tray placement and removal steps of dissolution testing. The robot arm 110 may be controlled by the processor of the dissolution testing system, which may require programming to control the movement of the robot arm 110, which programming and control is ascertainable by those skilled in the art to which the invention pertains in view of the disclosure herein.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention. All of the prior art identified above is incorporated by reference herein.

1. A dissolution testing system, comprising:
a frame for supporting a dissolution vessel having an interior into which dissolution media and a pharmaceutical compound are placed to conduct a dissolution test of the pharmaceutical compound;
a sampling probe movable into a position at least partly in an interior of the vessel;
temperature sensing means in connection with said sampling probe for sensing temperature of the dissolution media in the interior of the vessel when positioned below an upper surface of the dissolution media in the interior of the vessel;
imaging means in connection with said sampling probe for imaging dissolution of the pharmaceutical compound during the dissolution test;
media delivery means on said frame for delivering dissolution media to the interior of the vessel;
washing means on said frame for delivering washing media to the interior of the vessel to wash the interior of the vessel; and
a rotatable vessel housing to which the vessel is fixed, said vessel housing being rotatable between a first, testing position in which an opening of the vessel is oriented upward, and at least one additional position in which the opening is oriented downward to allow for fluid flow out of the interior of the vessel.

2. The system of claim 1, wherein said sampling probe comprises:
a hollow tube,
said temperature sensing means comprising a temperature sensor in a first end of said tube and configured to obtain a temperature of the dissolution media in the interior of the vessel when positioned below an upper surface of the dissolution media in the interior of the vessel,
said imaging means comprising a camera at the first end and configured to image dissolution of a pharmaceutical compound when subject to a dissolution test in the dissolution media in the vessel.

3. The system of claim 2, wherein said sampling probe further comprises an electrical slip ring at a second end of said tube opposite to the first end and configured to avoid tangles of at least one wire leading to said temperature sensor and said camera at a top of said tube.

4. The system of claim 2, wherein said sampling probe further comprises a cylindrical fixture in an interior of said tube to close a lower opening of said interior of said tube, said temperature sensor being on a side of said fixture inside of said tube and said camera being on said fixture and extending below a lower end of said tube.

5. The system of claim 1, wherein the vessel is part of the system, further comprising heating means outside of the interior of said vessel for heating a wall of said dissolution vessel to thereby cause the dissolution media in the interior of said dissolution vessel to be heated.

6. The system of claim 5, wherein said heating means comprise:
a first heating element below said vessel;
a second, intermediate heating element around a lower portion of said vessel; and
a third, upper heating element around an intermediate portion of said vessel above said second heating element.

7. The assembly of claim 6, further comprising a temperature control sensor that senses temperature of said wall of said vessel and controls heating provided by said first, second and third heating elements to prevent overheating.

8. The system of claim 1, further comprising a self-adjusting vessel cover configured to freefall onto the vessel.

9. The system of claim 8, wherein said media delivery means comprises a conduit extending through said cover and a nozzle configured to be positioned in the interior of the vessel, and said washing means comprise a conduit extending through said cover and a washing nozzle configured to be positioned in the interior of the vessel.

10. The system of claim 8, wherein the pharmaceutical compound is a tablet, further comprising a tablet dropping tray from which tablets are dropped into the interior of the vessel, said cover being underneath said tablet dropping tray.

11. The system of claim 1, further comprising a vessel housing rotation mechanism coupled to said vessel housing and configured to rotate said vessel housing between the first and second positions, said rotation mechanism comprising a motor having an output shaft, a gear mounted to said vessel housing, and a transmission belt that transfers rotational motive force from said output shaft through said transmission belt to said gear.

12. The system of claim 11, wherein said at least one additional position includes an emptying position in which the opening of the vessel is to one side, and a washing position in which the vessel is upside down and the opening of the vessel is downward.

13. The system of claim 11, wherein said at least one additional position includes a washing position in which the vessel is upside down and the opening of the vessel is downward, the system further comprising a washing device for cleaning the interior of the vessel, said washing device comprising an extendable rotation nozzle arranged below the vessel.

14. The system of claim 13, wherein said washing device further comprises a telescoping extension mechanism coupled to said nozzle to extend upward upon flow of water through said rotation nozzle.

15. The system of claim 13, wherein said washing device further comprises a screen tray around said nozzle.

16. The system of claim 11, wherein said at least one additional position includes a drying position in which the opening of the vessel is angled downward to orient the opening of the vessel, further comprising a drying device that dries the interior of the vessel when the vessel is in the drying position.

17. The system of claim 1, further comprising a rotation carousel on said frame, said rotation carousel including a plurality of basket compartments each having an upper part for storing a respective, clean basket, a lower part configured to retrieve a respective, used basket and an intermediate part including at least one gasket, whereby to provide a new basket on said sampling probe, said sampling probe is movable into engagement with one of the baskets when in said upper part of a respective one of said compartments until such movement is resisted by said at least one gasket, and to remove a used basket from said sampling probe, said sampling probe is movable into said lower part of a respective one of said compartments until such movement is resisted by said at least one gasket and then moved out of said lower part of the respective one of said compartments such that the used basket remains in said lower part of the respective one of said compartments, and after which said carousel is rotated to enable said sampling probe to engage with a new basket in another one of said compartments.

18. The system of claim 1, wherein the pharmaceutical compound is a tablet, further comprising a rotatable tablet loading device arranged above the vessel and including a rotation carousel configured to contain from six to twelve tablet compartments arranged around a periphery of said carousel, and a gate below said carousel and through which a tablet falls, said gate being controlled relative to rotation of said carousel to control dropping of tablets into the vessel.

19. The system of claim 1, further comprising an automatic filter tip replacement device comprising a movable filter track configured to receive a row of filter tips and situated above the vessel, and a gate below said filter track, said gate having a closed position in which a sampling probe is moved into engagement with one of the filter tips on said filter track and an open position in which the sampling probe passes through said gate with an engaged filter tip into the interior of the vessel, said filter track being moved to alternatingly bring each of the filter tips into a position to be engaged by the sampling probe.

20. The system of claim 1, further comprising a sample tray transfer station including a movable robot arm, a sample tray storage rack on one side of said robot arm in which a plurality of storage racks are stored, and a sample collector on another side of said robot arm in which samples are collected, said robot arm being controlled to individually place one of said storage racks from said sample try storage rack onto said sample collector to collect samples, and remove each of said storage racks from said sample collector after samples are collected and move it to said sample tray storage rack.
